(19) United States
(12) Patent Application Publication
(10) Pub. No.: US 2016/0206339 A1
(43) Pub. Date: Jul. 21, 2016

(54) SURGICAL INSTRUMENT

(71) Applicant: SMITH & NEPHEW, INC., Memphis, TN (US)

(72) Inventors: Mireille Akilian, Somerville, MA (US); Peter Cesarini, Londonderry, NH (US)

(21) Appl. No.: 14/912,259

(22) PCT Filed: Aug. 15, 2014
(86) PCT No.: PCT/US2014/051315

§ 371 Date: Feb. 16, 2016

Related U.S. Application Data
(60) Provisional application No. 61/866,563, filed on Aug. 16, 2013.

(51) Int. Cl. A61B 17/3205

(52) U.S. Cl. CPC A61B 17/3205

(57) ABSTRACT

A surgical instrument. Example surgical instruments include a cutting member being at least partially positioned within the outer member. A drive member coupled to the cutting member such that in response to only a rotational force applied to the drive member in a single direction, the cutting member (i) rotates and (ii) moves linearly in a back-and-forth motion. The drive member having a continuous helical groove including a first helical channel and a second helical channel that are blended at their ends. The first helical channel having a first helical angle that causes the cutting member to move in a first direction at a first linear speed and the second helical channel having a second helical angle that is smaller than the first helical angle that causes the cutting member to move in a second opposing direction at a second linear speed that is slower than the first linear speed.

### CROSS-REFERENCE TO RELATED APPLICATIONS

This application claims the benefit of Patent Cooperation Treaty (PCT) Application No. PCT/US2014/051315 filed Aug. 15, 2014, titled “Surgical Instrument,” and also claims the benefit of U.S. Provisional Application No. 61/866,563 filed Aug. 16, 2013. Both of these documents are incorporated by reference herein as if reproduced in full below.

### FIELD

This disclosure generally relates to surgical instruments, and more particularly, to surgical instruments that include a cutting element.

### BACKGROUND

Conventional arthroscopic surgical instruments generally include an outer tube and an inner member that rotates or moves linearly within the outer tube. The outer tube and inner member may interact to create shear forces that cut tissue. This type of cutting is generally used to cut soft tissue, such as muscle, ligaments, and tendons.

### BRIEF DESCRIPTION OF THE DRAWINGS

FIG. 1A is a partial top view of a surgical instrument according to some implementations of the present disclosure;

FIG. 1B is a cross-sectional view taken along 1B-1B in FIG. 1A;

FIG. 2A is a top view of an inner drive hub of the surgical instrument of FIG. 1;

FIG. 2B is a cross-sectional view taken along 2B-2B of the inner drive hub of FIG. 2A;

FIG. 2C is a distal end view of the inner drive hub of FIG. 2A;

FIG. 2D is a proximal end view of the inner drive hub of FIG. 2A;

FIG. 3A is a top view of a helical member of the surgical instrument of FIG. 1;

FIG. 3B is a side view of the helical member of FIG. 3A;

FIG. 3C is a cross-sectional view taken along 3C-3C of the helical member of FIG. 3A;

FIG. 3D is a proximal end view of the helical member of FIG. 3A;

FIG. 4A is a top view of an outer hub of the surgical instrument of FIG. 1;

FIG. 4B is a cross-sectional view taken along 4B-4B of the outer hub of FIG. 4A;

FIG. 4C is a distal end of the outer hub of FIG. 4A;

FIG. 5A is an exploded perspective view of a coupling piece and the helical member of the surgical instrument of FIG. 1;

FIG. 5B is an assembled partial cutaway view of the coupling piece and the helical member shown in FIG. 5A;

FIG. 5C is an assembled side view of the coupling piece and the helical member shown in FIG. 5A;

FIG. 5D is an assembled perspective view of coupling piece and the helical member shown in FIG. 5C;

FIG. 6A is a side view of a follower of the coupling piece of the surgical instrument of FIG. 1;

FIG. 6B is a cross-sectional view taken along 6B-6B of the follower of FIG. 6A;

FIG. 6C is a top view of the follower of FIG. 6A;

FIG. 7A is a top view of a cap of the follower of the coupling piece of the surgical instrument of FIG. 1;

FIG. 7B is a cross-sectional view taken along 7B-7B of the cap of FIG. 7A;

FIG. 8A is a partial top view of an outer member of the surgical instrument of FIG. 1;

FIG. 8B is a partial side view of the outer member of FIG. 8A;

FIG. 9 is a partial side view of an inner member of the surgical instrument of FIG. 1;

FIG. 10 illustrates the surgical instrument of FIG. 1 in use to cut tissue;

FIG. 11 is a partial side view of an alternate implementation of an inner member of a surgical instrument according to some implementations of the present disclosure; and

FIG. 12 is a partial perspective view of an alternate implementation of a helical member of a surgical instrument according to some implementations of the present disclosure.

While the invention is susceptible to various modifications and alternative forms, a specific implementation thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit of the invention.

### DESCRIPTION

As described above, surgical instruments may include an outer tube and an inner member, where the inner member moves relative to the outer tube to create shear forces that are employed to cut tissue. According to various aspects disclosed herein, embodiments of surgical instruments are configured to achieve desired relative movement between the outer tube and the inner member. According to various aspects disclosed herein, the inner member moves linearly at different rates depending on its position and/or direction of movement relative to the outer tube. Thus, in such embodiments, the surgical instruments are configured to provide a back-and-forth linear motion that increases their cutting performance and/or other aspects of their operation.

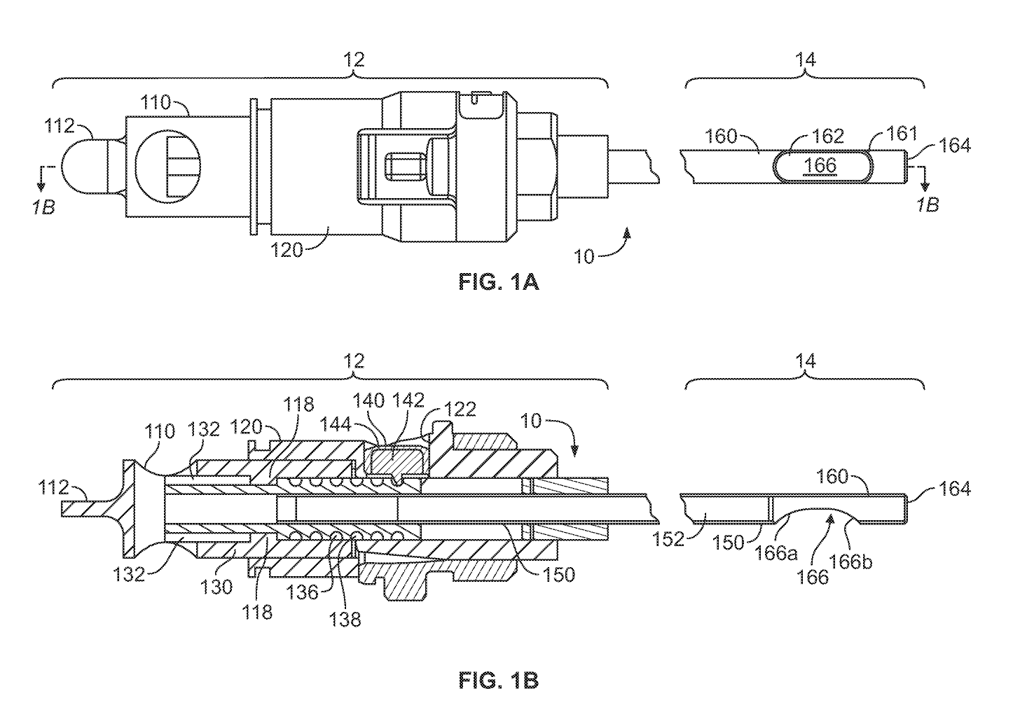

In some embodiments, the inner member is rotated relative to the outer tube and the rotation causes the inner member also to move linearly and back-and-forth relative to the outer tube. As an example, a cutting device 10 is described with reference to FIGS. 1-11. As shown in FIGS. 1A and 1B, the cutting device 10 includes a driving end 12 and a cutting end 14. The driving end 12 is located at the proximal end of the cutting device 10. The cutting end 14 is located at the distal end of the cutting device 10.

At the driving end 12, there is an inner drive hub 110 and an outer hub 120. The inner drive hub 110 includes a drive coupler 112, which mounts into a rotary driver (not shown). The rotary driver turns the drive coupler 112 causing a helical member or drive member 130 and the inner drive hub 110 to rotate. The helical member or drive member 130 is located within the inner drive hub 112 and the outer hub 120. The helical member or drive member 130 and a coupling piece 140 engage each other so that rotation of the helical member or drive member 130 causes linear motion of the helical member or drive member 130.

The cutting device 10 includes an elongated inner member or cutting member 150 and an elongated outer member 160, as shown in FIG. 1B. The inner member or cutting member 150 is tubular with a hollow interior 152 and used to cut or slice/shear tissue. The inner member 150 is coupled to the helical member 130 to enable linear and rotary motion of the inner member 150.

The outer member 160 is also tubular with a hollow interior 162. The inner member 150 is received inside the outer member 160. The outer member 160 is coupled to the outer hub 120. The outer member 160 may include a tip 164, which is blunt, e.g., the corners are rounded. At the cutting end 14, the outer member 160 defines a cutting window 166 through a wall 161 (FIG. 1A) of the outer member 160.

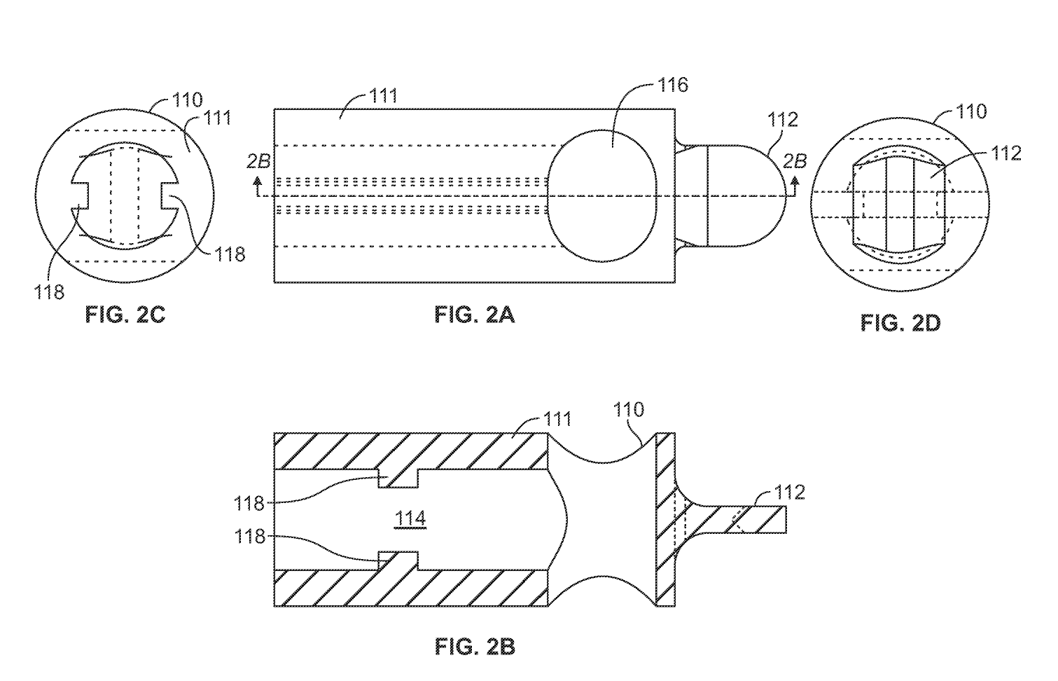

Referring to FIGS. 2A-2D, the inner drive hub 110 may include the drive coupler 112 (FIGS. 2A, 2B, 2D), a lumen 114 (FIG. 2B), an aspiration opening 116 (FIG. 2A), and at least one key 118 (FIGS. 2B and 2C). Debris from the cutting end 14 (FIGS. 1A and 1B) of the cutting device 10 may be aspirated through the aspiration opening 116. The drive coupler 112 extends from the proximal end of the inner drive hub 110 and couples the inner drive hub 110 to the rotary driver (not shown). The rotary driver may include a drive motor that is coupled to the drive coupler 112 to cause the drive hub 110 to rotate. The drive hub 110 transfers rotary motion to the helical member 130 while allowing the inner member 150 (which is coupled to the helical member) to move axially along the axis of rotation.

At least one key 118 extends from a wall 111 of the inner drive hub 110. Each key 118 functions as a guide along one side of the inner drive hub 110. Each key 118 of the inner drive hub 110 engages a respective slot 132 of the helical member 130 so that rotation of the inner drive hub 110 causes the helical member 130 to rotate while allowing the helical member 130 to move linearly relative to the inner drive hub 110, e.g., each key 118 slides linearly along the respective slot 132. As shown in FIGS. 1B and 2B-2D, the at least one key 118 is shaped like a fin and the at least one slot 132 is located at the proximal end of the helical member 130 to receive the at least one key 118 of the inner drive hub 110. In alternative implementations, the at least one slot is conversely disposed in the wall 111 of the inner drive hub 110 while the at least one key extends from a wall of the helical member 130 to engage the at least one slot. In the illustrated implementations, a pair of keys 118 engages respective slots 132. In general, however, any number of keys 118 may extend from inner drive hub 110 to engage respective slots 132 in the wall of the helical member 130, or vice versa. In alternative implementations, the rotary driver (not shown) may be coupled to the helical member 130 by gears or a gear and a spline gear.

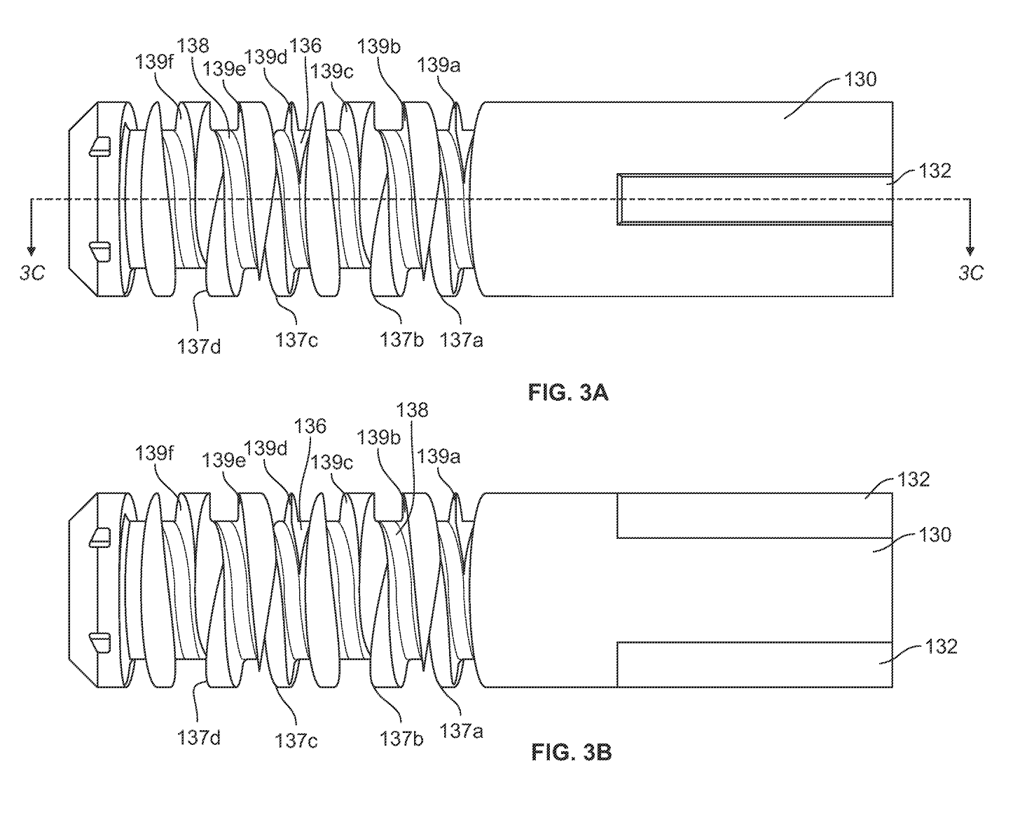

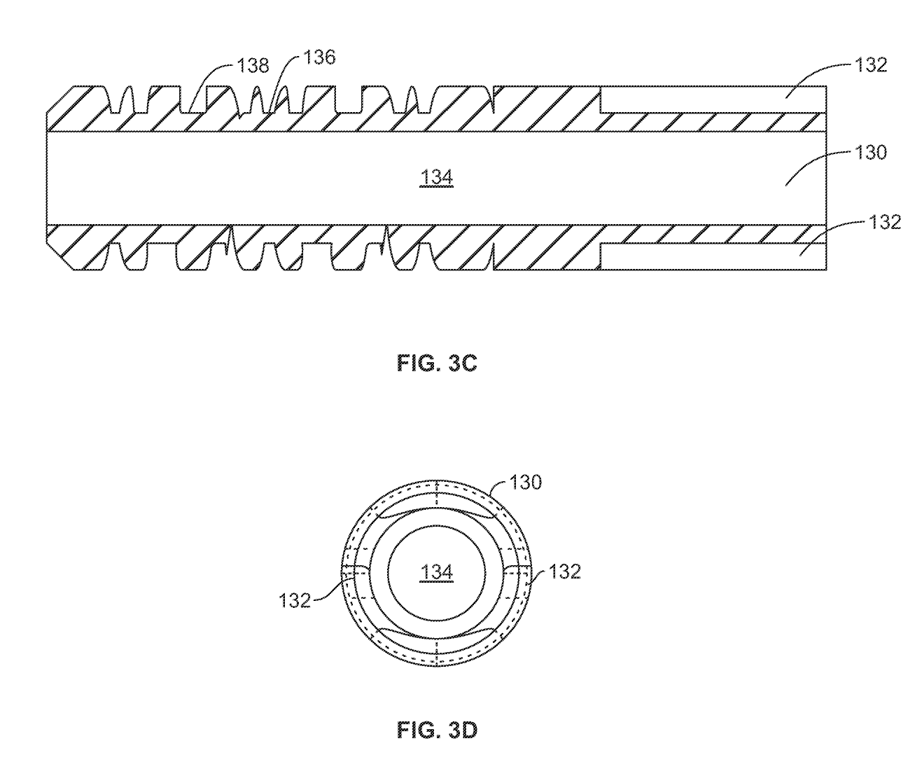

Referring to FIGS. 3A-3D, the helical member 130 of the cutting device 10 is formed of a material in a tubular shape with a lumen 134 (FIGS. 3C and 3D). The inner member 150 may be disposed within the lumen 134 of the helical member 130 and fixed therein, for example, by set screws, epoxy, injection-molded, or over-molded plastic. In alternative implementations, the inner member 150 may be coupled to the helical member 130 by a spline, gears or a gear and a spline.

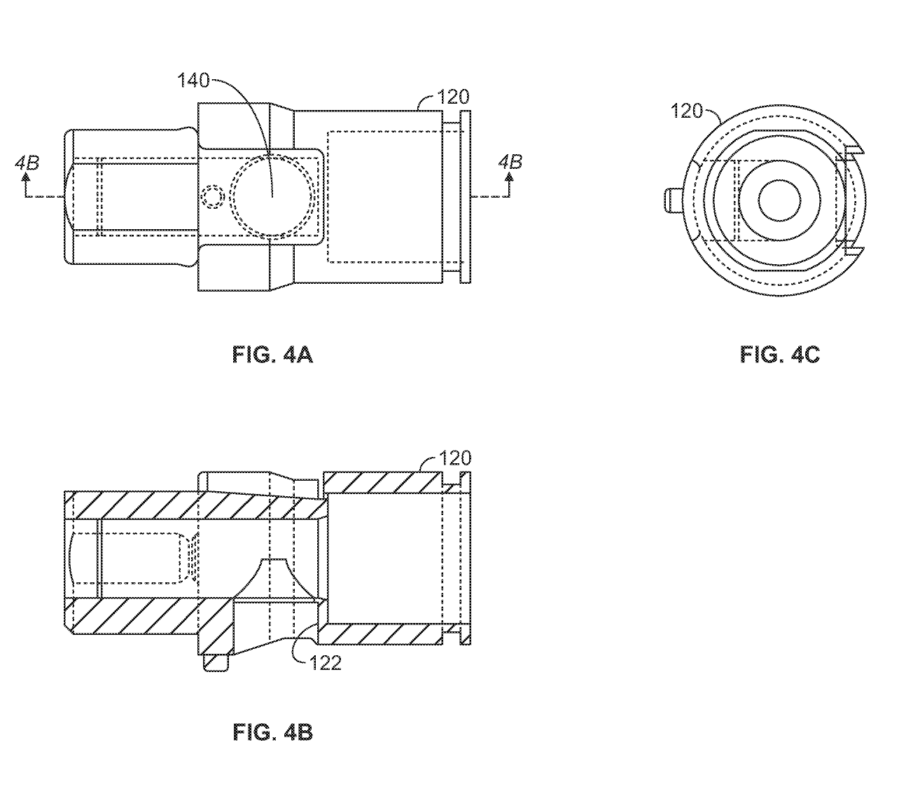

Referring to FIGS. 4A-4C, the outer hub 120 of the cutting device 10 is formed of hard plastic and does not move. A cutout 122 (FIG. 4B) is disposed within a wall of the outer hub 120, for example, centrally, as in FIG. 4B, and aligned with the helical member 130. As shown in FIG. 1B, the coupling piece 140 is located in the cutout 122 of the outer hub 120.

As shown in FIG. 1B, the outer member 160 is disposed within the outer hub 120 and fixed therein by a coupling using, for example, set screws, epoxy, glue, insert molding, or spin-welding.

Referring particularly to FIGS. 3A-3C, the helical member 130 also includes two helical channels 136, 138. The helical channels 136, 138 are disposed on a distal portion of the exterior surface of the helical member 130. As shown, the helical channel 136 is right-hand threaded; the other helical channel 138 is left-hand threaded. The length of the distal portion of the helical member 130 with helical channels 136, 138 may be longer, shorter, or the same length as the length of the cutting window 166 (FIGS. 1A and 1B). The helical channels 136, 138 may be smoothly blended together at their ends to form a continuous groove so that there is a smooth transition from one helical channel to the other helical channel at each end of the distal portion of the helical member 130. The continuous groove provides for linear motion of the inner member 150 that includes moving distally over a length of travel and then changing direction and moving proximally over a length of travel and then changing direction to begin moving distally again. The length of travel can be determined as a function of the extent of the helical channels 136, 138 over the helical member 130. The velocity of the linear motion can be determined as a function of the angle or pitch of the helical channels 136, 138 and the rotational speed of the helical member 130. Changing direction includes, while moving in a first direction, decelerating to zero velocity and then accelerating in the opposite direction.

In accordance with some implementations of the present disclosure, the helical member 130 may be mechanically driven by the rotary driver (not shown) and moves linearly over a length of travel and then changes direction as a result of the interaction of the coupling piece 140 with the helical channels 136, 138. In such implementations, only a rotational force in a single rotational direction applied by the rotary driver to the helical member 130 is needed to drive the helical member 130. By drive the helical member 130 it is meant that the helical member is caused to rotate and move linearly in a back-and-forth motion. In accordance with other implementations of the present disclosure, the helical member 130 may be mechanically driven by the rotary driver and the coupling piece 140 moves linearly over a length of travel and then changes direction as a result of the interaction of the coupling piece 140 with the helical channels 136, 138. The coupling piece 140 can be coupled to the inner member 150 and cause the inner member 150 to move linearly and then change direction.

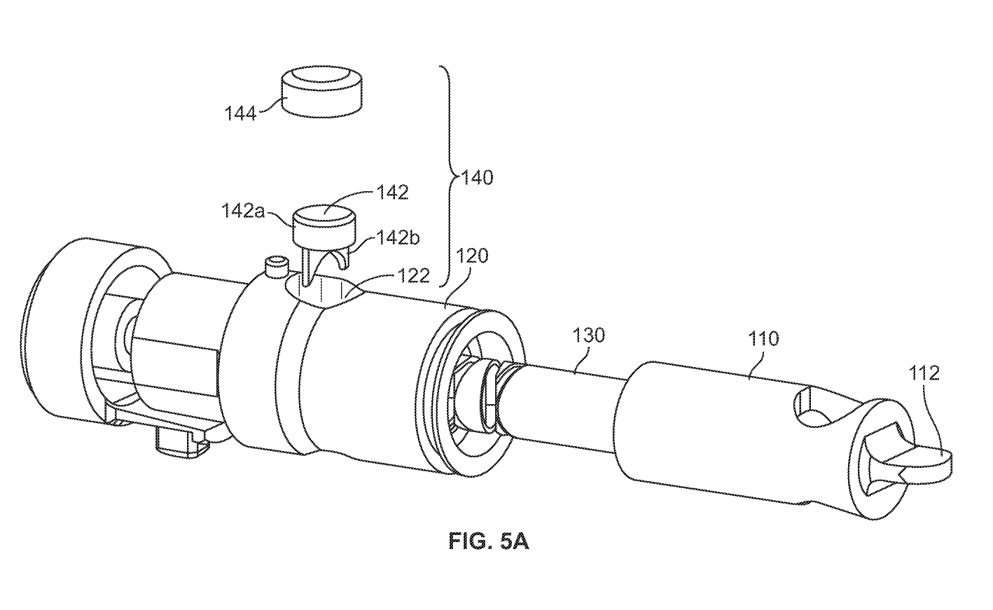

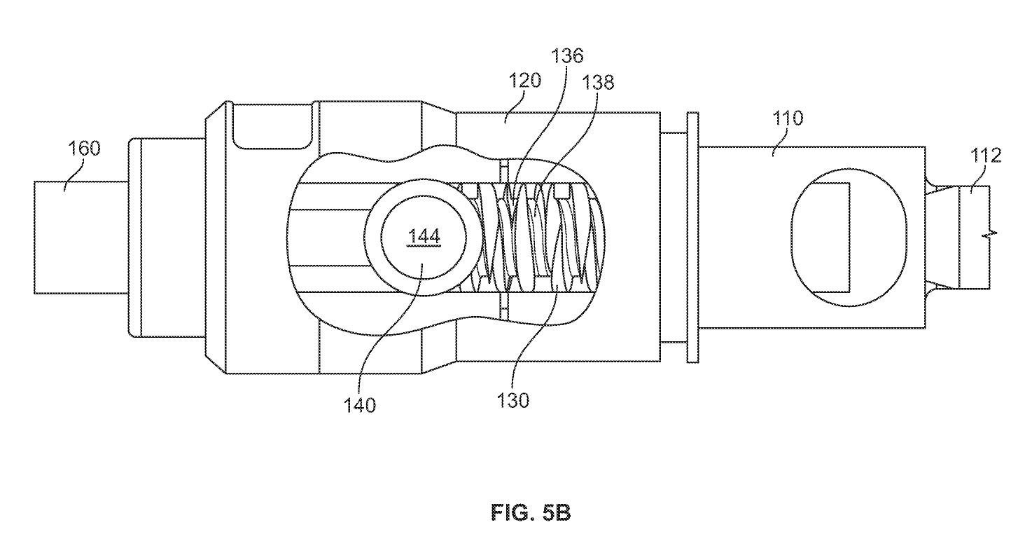

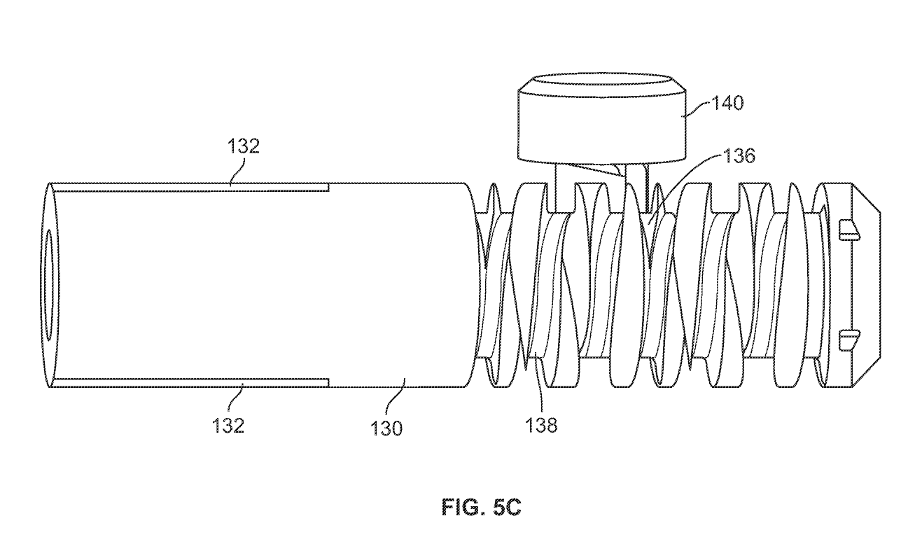

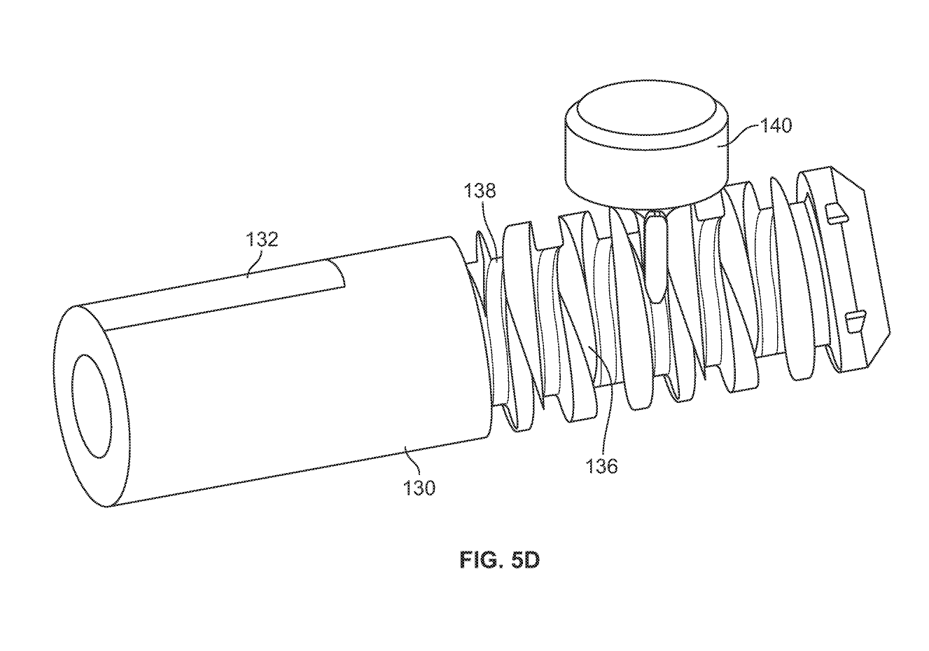

Referring to FIG. 5A, the coupling piece 140 includes a follower 142 and a cap 144. Having the two helical channels 136, 138 that are smoothly blended together at their ends to form a continuous groove in conjunction with the slot 132/key 118 coupling of the inner drive hub 110 and the helical member 130, the rotary driver only needs to rotate in a single direction and does not require reversal of the rotational direction upon the coupling piece 140 reaching the end of one of the helical channels 136, 138. That is, the helical member 130 is caused to move distally in a first direction and then in a second opposite direction without having to change the rotational direction of the rotary driver.

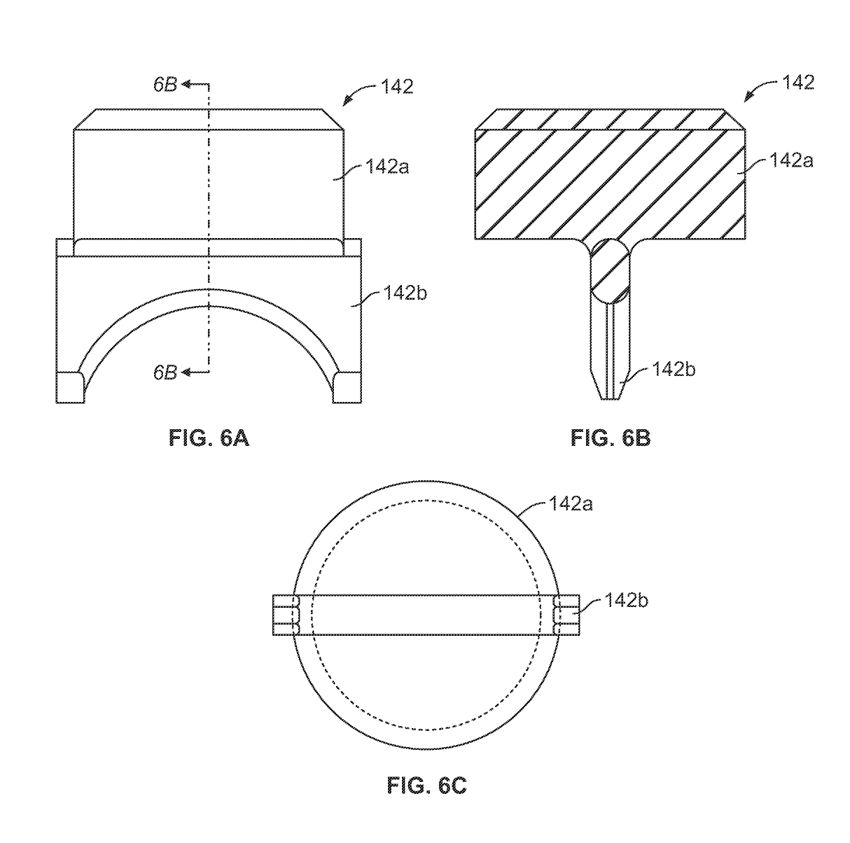

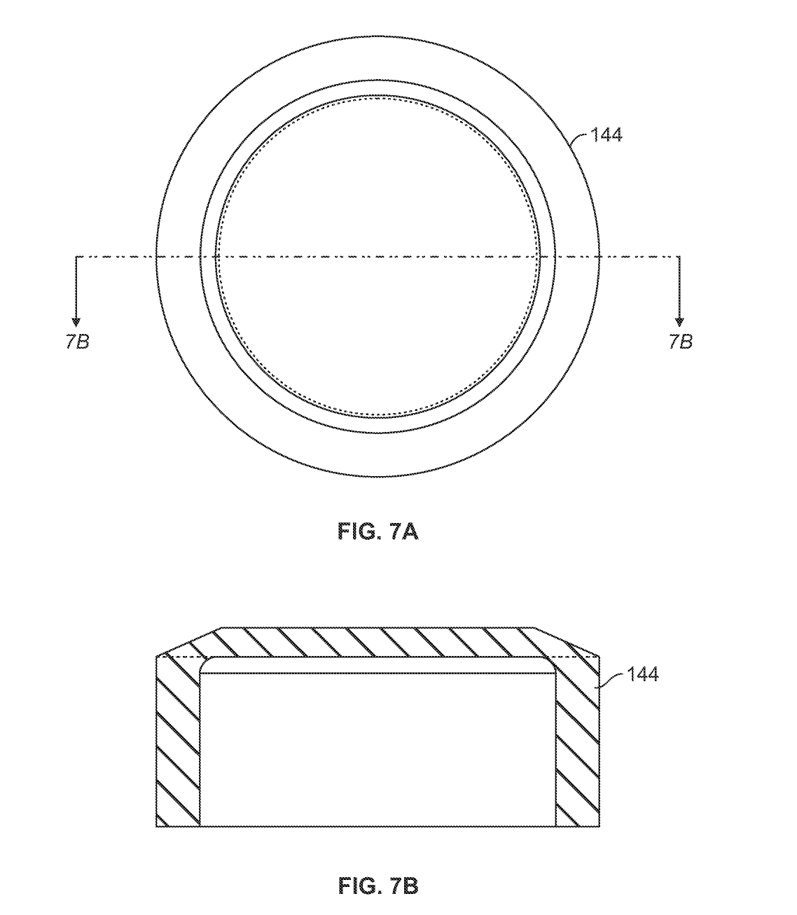

Referring to FIGS. 6A-6C, the follower 142 includes a cylindrical head 142a and two legs 142b. As shown in FIGS. 5B-5D, the legs 142b form an arch and rest in the channels of the helical channels 136, 138 formed in the distal portion of the exterior surface of the helical member 130. The arch of the legs 142b is dimensionally related to the diameter described by the helical channels 136, 138 of the helical member 130. Referring to FIGS. 7A and 7B, the cap 144 of the coupling piece 140 is shown, which, as best shown in the partially exploded view of FIG. 5A, covers the follower 142 to provide a seal to allow sufficient suction to remove aspirated debris. Also, the cap 144 may be a separate piece from the follower 142 in order to allow the follower 142 to swivel.

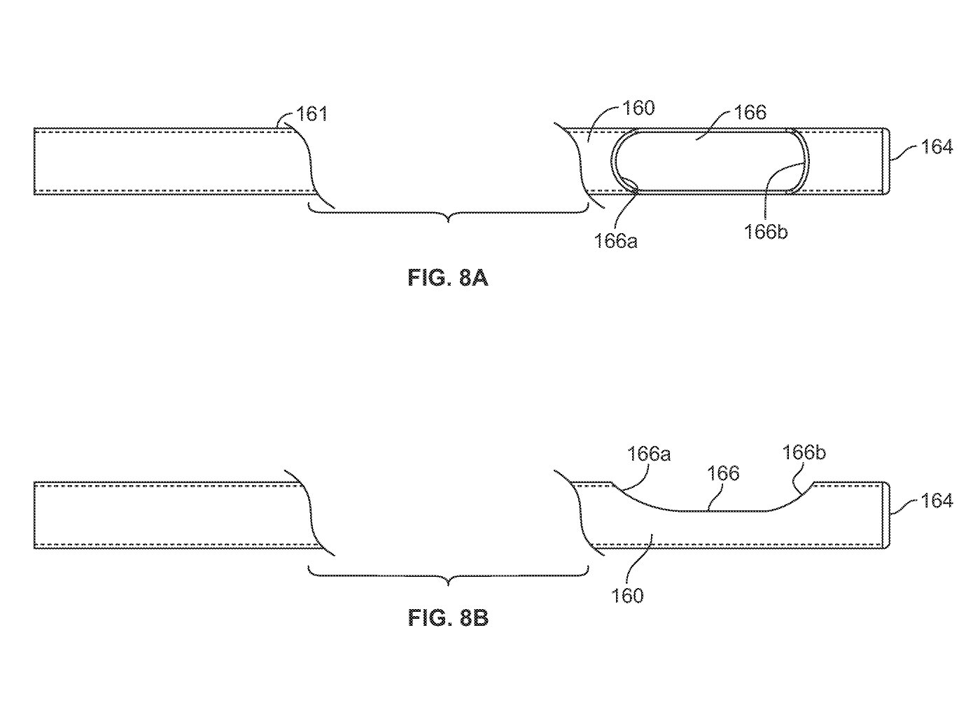

As shown in FIGS. 8A and 8B, the cutting window 166 has a generally oblong shape and is disposed proximate to the tip 164 of the outer member 160 and along the length of the outer member 160 from the distal tip 164 to a position proximate the helical member 130. The cutting window 166 exposes the inner member 150 over a length. The proximal end 166a of the cutting window 166 is U-shaped. The distal end 166b of the cutting window 166 is also U-shaped. It is understood, however, that the cutting window 166 may be shaped and/or positioned in a manner different from the illustrated embodiments. In some embodiments, the distal end 166b may optionally provide a sharp edge. In further embodiments, the distal end 166b may optionally have a saddle shape that forms a hook, which may pierce the targeted tissue to hold the tissue as the inner member 150 cuts.

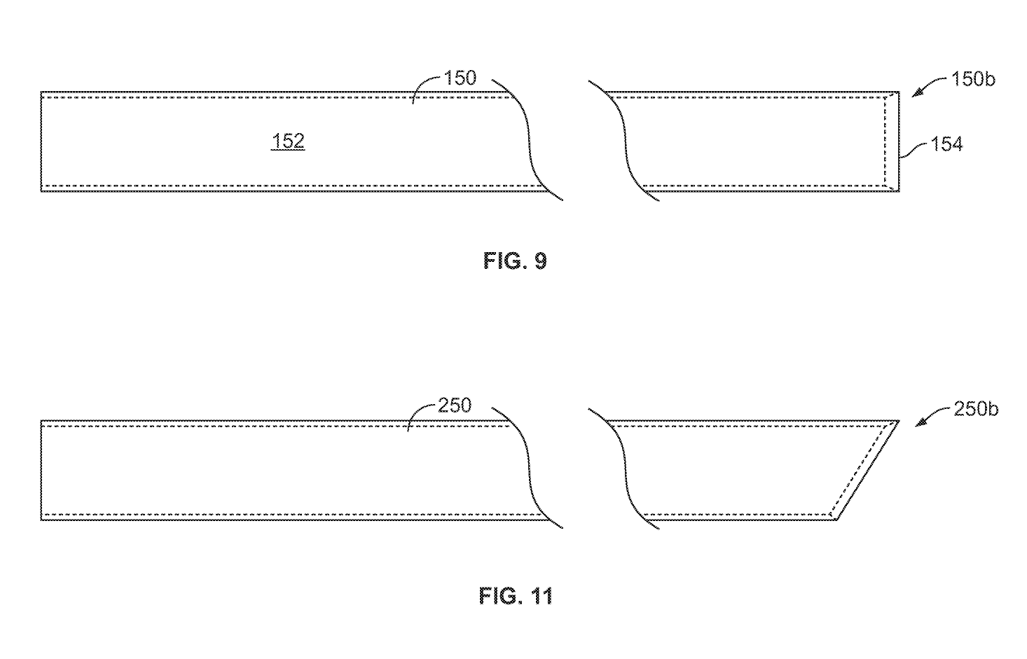

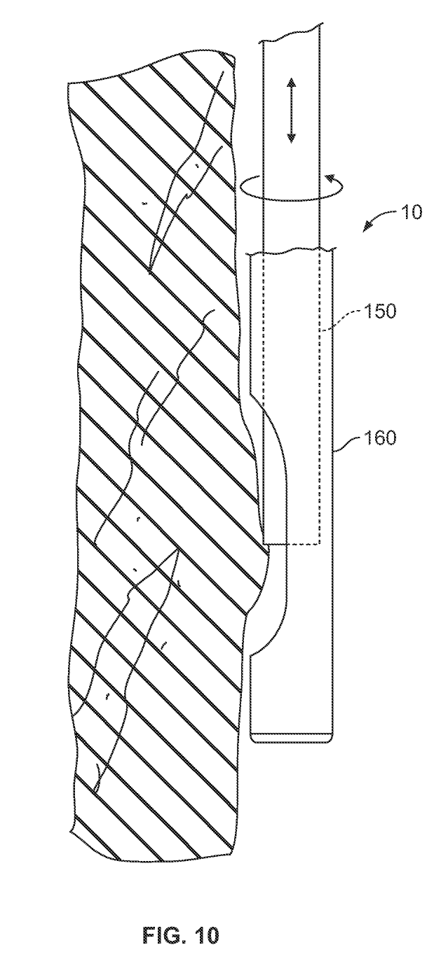

FIG. 9 shows that the inner member 150 is generally tubular with a hollow interior 152. Aspiration of debris occurs through the hollow interior 152 of the inner member 150, and through the lumen 134 (FIGS. 3C and 3D) of the helical member 130 to the aspiration opening 116 (FIG. 2A) of the inner drive hub 110. The distal end 150b of the inner member 150 is chamfered to a sharp edge 154 for cutting. The inner member 150 simultaneously rotates about its axis and moves linearly along its axis of rotation to cut tissue. The cutting surface of the distal end 150b of the inner member 150 shears tissue. For example, referring to FIG. 10, the cutting device 10 is placed tangentially against the targeted tissue such that the cutting window 166 exposes the inner member 150 to the tissue. The tissue protrudes through the cutting window 166 prior to being cut by the inner member 150. As the inner member 150 rotates and moves linearly (e.g., downward in the orientation shown in FIG. 10), as shown by the arrows, the cutting edge 154 of the inner member 150 shears the tissue as the inner member 150 advances to cut the tissue. The cut is completed as the cutting edge 154 (FIG. 9) of the inner member 150 advances beyond the distal end 166b (FIGS. 8A and 8B) of the cutting window 166 within the outer member 160.

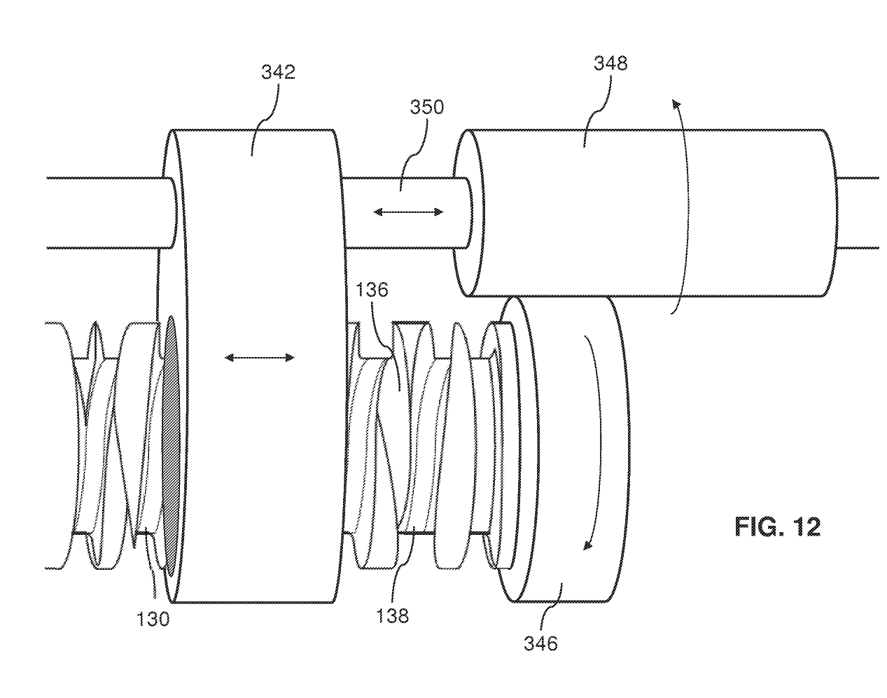

FIG. 11 shows an alternative implementation of the inner member. The distal end 250b of the inner member 250 may be angled to a chamfered point so that the cut in the targeted tissue is initiated on one side and then extends across the width of the tissue. Similarly, when the cutting device is placed tangentially against the targeted tissue, the rotating and linearly moving inner member 250 shears the tissue to be cut.

Referring particularly to FIGS. 5C and 5D, as the helical member 130 and the inner drive hub 110 (FIGS. 2A-2D) are mechanically driven by the rotary driver (not shown), the follower 142 (FIGS. 6A and 6B) of the coupling piece 140 follows the helical channels 136, 138, swiveling as the follower 142 smoothly transitions from helical channel 136 to helical channel 138 at the ends of the distal portion of the helical member 130 having the helical channels 136, 138. The coupling of the follower 142 to the helical channels 136, 138 causes the helical member 130 to also move linearly. Thus, the inner member 150 simultaneously rotates and moves linearly to cut the tissue.

When the helical member 130 moves toward the distal end, the cutting edge 154 (FIG. 9) of the inner member 150 advances and closes the cutting window 166 (FIGS. 8A and 8B) so that the cutting device 10 engages and cuts targeted tissue. Meanwhile, when the helical member 130 moves away from the distal end, the cutting edge 154 withdraws and opens the cutting window 166 so that the resulting debris can be aspirated through the window 166 and into the hollow interior 152 of the inner member 150. In addition, the opening of the cutting window 166 allows tissue to be drawn in for the next cut. Because the cutting device 10 performs different operations (e.g., cutting, aspirating, etc.) when the helical member 130 moves toward or away from the distal end, it may be advantageous to optimize the movement of the helical member 130 according to the different functions. For example, in some cases, it may be advantageous to move the helical member 130 more slowly away from the distal end and thus keep the cutting window 166 open for a relatively longer period of time after a cut to allow sufficient aspiration of debris and to provide time for more tissue to enter the cutting window 166 in preparation for the next cut. Accordingly, aspects of the present disclosure provide a helical device that is configured to move linearly at different rates depending on its position and/or direction of movement.

The legs 142b (FIG. 6A) of the follower 142 of the coupling piece 140 travel over the helical channels 136, 138 to produce the desired linear motion of the helical member 130 from the input rotary motion. Movement of the coupling piece 140 along the first helical channel 136 causes the helical member 130 to move toward the distal end and causes the cutting device to perform the cutting operation. Meanwhile, movement of the coupling piece 140 along the second helical channel 138 causes the helical member 130 to move away from the distal end and allows the cutting device to perform the aspirating operation and draw more tissue into the cutting window 166 for cutting.

The first helical channel 136 is defined by a thread with a first helical angle or pitch. As shown in FIGS. 3A-3B, the thread for the first helical channel 136 defines four turns 137a-d distributed evenly along a distance of the helical member 130. Meanwhile, the second helical channel 138 is defined by a thread with a second helical angle or pitch. The thread for the second helical channel 138 defines six turns 139a-f distributed evenly along the same distance of the helical member 130. Thus, the first helical channel 136 has fewer turns than the second helical channel 138 over the same distance. The number of turns over a distance generally corresponds to the number of rotations required by the helical member 130 to travel linearly over the same distance. To define more turns over a given distance of the helical member 130, a thread must generally have a smaller helical angle or pitch. Thus, the second helical angle or pitch associated with the second helical channel 138 is smaller than the first helical angle or pitch associated with the first helical channel 136.

To move linearly over a given distance, the helical member 130 must make fewer rotations when the coupling piece 140 travels over the first helical channel 136, i.e., when the helical member 130 moves toward the distal end. Conversely, the helical member 130 must make more rotations when the coupling piece 140 travels over the second helical channel 138, i.e., when the helical member 130 moves away from the distal end. When the helical member 130 is rotated at a constant speed, (1) the helical member 130 moves at a relatively faster linear speed when it is moving toward the distal end to perform the cutting operation and (2) conversely, the helical member 130 moves at a relatively slower linear speed when the helical member 130 is moving away from the distal end to perform the aspirating operation and draw more tissue into the cutting window 166 for subsequent cutting.

The first helical channel 136 may be configured with a particular helical angle or pitch so that the cutting device performs the cutting operation at a particular linear speed for optimal performance. Meanwhile, the second helical channel 138 may be configured with a relatively smaller helical angle or pitch to keep the cutting window 166 at least partially open for a longer time. As described above, it may be advantageous to move the helical member 130 more slowly away from the distal end and thus keep the cutting window 166 open for a longer period of time after a cut. This allows for sufficient aspiration of debris and provides time for more tissue to enter the cutting window 166 in preparation for the next cut.

Although the thread for the first helical channel 136 defines four turns 137a-d and the thread for the second helical channel 138 defines six turns 139a-f over the same linear distance of the helical member 130, it is understood that, in other embodiments, the first helical channel and the second helical channel may be configured with different respective helical angles so they have different respective numbers of turns than shown in FIGS. 3A-3C. In addition, although the entire length of the first helical channel 136 may be defined by the first helical angle or pitch and the entire length of the second helical channel 138 may be defined by the second helical angle or pitch in FIGS. 3A-3C, it is understood that other implementations may employ one or more helical channels that are defined by multiple helical angles or pitches that cause the helical member to move at various different speeds as the helical member moves in one direction. Furthermore, it is understood that in alternative implementations, the helical channels may be configured so that the first helical channel has a smaller helical angle than the helical angle of the second helical channel, thereby causing the helical member to move relatively slower toward the distal end (to perform the cutting operation more slowly) and relatively faster away from the distal end (to perform the aspirating operation, etc. more quickly).

The helical member 130 can be generally used in the cutting device 10 described above. It is understood, however, that aspects of the helical member 130 may be employed in other types of cutting devices to achieve corresponding advantages.

In general, according to some aspects of the present disclosure, surgical instruments employ a helical member with helical channels that are configured to provide optimal linear motion. The helical channels can be smoothly blended at their ends to provide a continuous channel that provides for a change in direction at the ends of the linear motion. In particular, the helical channels are defined by threads with different helical angles so that the rotation of the helical member causes linear movement at different desired linear speeds.

The resulting linear movement may involve relative movement between any components of the surgical instruments and are not limited to the examples and implementations described herein. The helical member may be fixed to a first component (e.g., in a housing) and rotation of the helical member relative to a second component also causes relative linear movement between the first and second components. In accordance with some implementations of the present disclosure, the helical member can be stationary and a follower can be permitted to move linearly along the helical member, such as in a carriage or guide. The follower can be coupled to the inner member to cause the inner member to move linearly as the follower is moved by rotational motion of the helical member. A separate drive train, such as gears, belts and pulleys, can be used to impart rotary motion on the inner member. In accordance with some implementations of the present disclosure, the inner member can rotate about the same axis, a parallel axis, or a non-parallel axis as the helical member.

For example, the helical member 130 shown in FIG. 12 is driven by a rotary driver (not shown), but unlike the helical member 130 in the implementations of FIGS. 1A-11, the helical member 130 in FIG. 12 does not move linearly together with inner member 350. Rather, the inner member 350 moves linearly relative to the helical member 130. For example, the helical member 130 may remain stationary relative to a housing element while the inner member 350 moves linearly relative to the housing element. As FIG. 12 shows, a follower 342 moves linearly along the helical channels 136, 138 of the helical member 130. The movement of the follower 342 is determined by the helical channels 136, 138 as described above. The follower 342 is coupled to the inner member 350, so that the inner member 350 also moves linearly along a parallel axis according to the helical channels 136, 138. In particular, the inner member 350 moves linearly at different rates depending on its position and/or direction of movement relative to an outer member (not shown). To cause rotation of the inner member 350, the helical member 130 is coupled to a gear 346 that engages a gear 348 coupled to the inner member 350. The gear 346 slides along, and remains engaged with, the longer gear 348 as the gear 348 moves linearly with the inner member 350. Rotation of the helical member 130 causes corresponding rotation of the gears 346, 348 to rotate the inner member 350. Accordingly, the helical member 130 causes linear and rotary movement of the inner member 350 relative to the outer member to produce the desired cutting operation, aspirating operation, etc.

While the present disclosure has been described with reference to one or more particular implementations, those skilled in the art will recognize that many changes may be made thereto without departing from the spirit and scope of the present disclosure. Each of these implementations and obvious variations thereof is contemplated as falling within the spirit and scope of the present disclosure, which is set forth in the following claims. It is also contemplated that additional implementations according to aspects of the present disclosure may combine any number of features from any of the implementations described herein.

1.-40. (canceled)

41. A surgical instrument, comprising:
an outer member defining a cutting window;
a cutting member at least partially positioned within the outer member, the cutting member including a sharp edge for cutting tissue associated with the cutting window; and
a drive member coupled to the cutting member such that in response to only a rotational force applied to the drive member in a single rotational direction, the cutting member (i) rotates and (ii) moves linearly in a back-and-forth motion, the drive member having a continuous helical groove including a first helical channel and a second helical channel, the first helical channel and the second helical channel being blended at their respective ends to form the continuous helical groove, at least a portion of the first helical channel having a first helical angle that causes the cutting member to move linearly in a first direction at a first linear speed and at least a portion of the second helical channel having a second helical angle that is different than the first helical angle that causes the cutting member to move linearly in a second opposing direction at a second linear speed that is different than the first linear speed.

42. The surgical instrument of claim 41, wherein the second helical angle is smaller than the first helical angle.

43. The surgical instrument of claim 42, wherein the second linear speed is slower than the first linear speed.

44. The surgical instrument of claim 41, wherein the second helical angle is larger than the first helical angle.

45. The surgical instrument of claim 44, wherein the second linear speed is faster than the first linear speed.

46. A method for driving a cutting member coupled to a drive member, the method comprising:
rotating the drive member in a single rotational direction, thereby rotating the cutting member in the same rotational direction;
the rotating the drive member further causing the cutting member to sequentially (i) move linearly in a first direction at a first linear speed, (ii) change directions from the first direction to a second opposing direction, (iii), move linearly in the second opposing direction at a second linear speed that is different than the first linear speed, and (iv) change directions back from the second direction to the first direction.

47. The method of claim 46, wherein second linear speed is slower than the first linear speed.

48. The method of claim 46, wherein second linear speed is faster than the first linear speed.

49. The method of claim 46, wherein the drive member has a continuous helical groove including a first helical channel and a second helical channel, the first helical channel and the second helical channel being blended at their respective ends to form the continuous helical groove.

50. The method of claim 49, wherein at least a portion of the first helical channel has a first helical angle that causes the cutting member to move linearly in the first direction at the first linear speed and at least a portion of the second helical channel has a second helical angle that is different than the first helical angle that causes the cutting member to move linearly in the second opposing direction at the second linear speed.

51. The method of claim 50, wherein the second helical angle is smaller than the first helical angle.

52. The method of claim 50, wherein the second helical angle is larger than the first helical angle.

53. The surgical instrument of claim 41, wherein the surgical instrument is configured to perform a cutting operation when the cutting member moves linearly in the first direction at the first linear speed and wherein the surgical instrument is configured to perform an aspirating operation when the cutting member moves linearly in the second direction at the second linear speed.

54. The surgical instrument of claim 53, wherein the surgical instrument is configured to transport tissue and fluid through the cutting window, through the cutting member, and through a lumen in the drive member.

55. The surgical instrument of claim 41, wherein the at least a portion of the first helical channel defines a first number of evenly distributed turns along a distance of the helical member and the at least a portion of the second helical channel defines a second number of evenly distributed turns along the same distance of the helical member, the second number being greater than the first number.

56. The surgical instrument of claim 41, wherein the first helical angle is a non-zero angle with respect to an axis of rotation of the drive member and wherein the second helical angle is a non-zero angle with respect to the axis of rotation of the drive member.

57. The surgical instrument of claim 56, wherein the first direction is parallel with the axis of rotation and wherein the second direction is parallel with the axis of rotation

58. The surgical instrument of claim 41, further comprising a coupling piece at least partially disposed in the continuous helical groove of the drive member such that rotation of the drive member causes the cutting member to move linearly in the back-and-forth motion.

59. The method of claim 46, wherein the drive member has a continuous helical groove including a first helical channel and a second helical channel, the first helical channel and the second helical channel blended at their respective ends to form the continuous helical groove.

60. The method of claim 46, wherein the causing the cutting member to sequentially move and change directions includes moving the cutting member within an outer member having a cutting window such that when the cutting member moves linearly in the first direction, the cutting member moves across the cutting window to close the cutting window, and when the cutting member moves linearly in the second direction, the cutting member withdraws to open the cutting window.
